(19) United States
(12) Patent Application Publication
(10) Pub. No.: US 2023/0010773 A1
(43) Pub. Date: Jan. 12, 2023

(54) SYSTEMS AND METHODS FOR GUIDING AN ULTRASOUND PROBE

(71) Applicant: KONINKLIJKE PHILIPS N.V., EINDHOVEN (NL)

(72) Inventors: Paul THIENPHRAPA, CAMBRIDGE, MA (US); Marcin Arkadiusz BALICKI, CAMBRIDGE, MA (US); William MCNAMARA, VALHALLA, NY (US)

(21) Appl. No.: 17/783,370

(22) PCT Filed: Dec. 4, 2020
(86) PCT No.: PCT/EP2020/084582

§ 371 Date: Jun. 8, 2022

Related U.S. Application Data
(60) Provisional application No. 62/947,167, filed on Dec. 12, 2019.

(51) Int. Cl. A61B 1/273; A61B 8/00; A61B 5/00

(52) U.S. Cl. CPC A61B 1/2733; A61B 8/4254; A61B 8/445; A61B 8/4416; A61B 5/0084; A61B 8/54

(57) ABSTRACT

An ultrasound device (10) comprises a probe (12) including a tube (14) sized for in vivo insertion into a patient and an ultrasound transducer (18) disposed at a distal end (16) of the tube. A camera (20) is mounted at the distal end of the tube in a spatial relationship to the ultrasound transducer. At least one electronic processor (28) is programmed to: control the ultrasound transducer and the camera to acquire ultrasound images (19) and camera images (21) respectively while the ultrasound transducer is disposed in vivo; construct keyframes (36) during in vivo movement of the ultrasound transducer, each keyframe representing an in vivo position of the ultrasound transducer and including at least ultrasound image features (38) extracted from at least one of the ultrasound images acquired at the in vivo position of the ultrasound transducer and camera image features (40) extracted from at least one of the camera images acquired at the in vivo position of the ultrasound transducer; generate a navigation map (45) of the in vivo movement of the ultrasound transducer comprising the keyframes; and output navigational guidance (49) based on comparison of current ultrasound and camera images acquired by the ultrasound transducer and camera with the navigation map.

### FIELD

The following relates generally to the ultrasound arts, ultrasound imaging arts, ultrasound probe arts, ultrasound probe guidance arts, ultrasound catheter arts, transesophageal echography (TEE) arts, and related arts.

### BACKGROUND

Ultrasound imaging employing an ultrasound transducer array mounted on the end of a catheter, and in particular transesophageal echocardiography (TEE), is an existing imaging methodology with various uses, most commonly for diagnostic purposes for cardiac patients and for providing image guidance during catheter-based cardiac interventional procedures. TEE involves an approach for cardiac ultrasound imaging in which the ultrasound probe includes a cable or tube with the ultrasound transducer located at its tip. The TEE probe is inserted into the esophagus to place the ultrasound transducers at its distal tip close to the heart.

Another use of TEE is for catheter-based structural heart interventions where TEE has been widely adopted as a reliable approach to imaging the interventional catheter instrument used in treating structural heart disease. Three-dimensional (3D) trans-esophageal ultrasound (US) is used for interventional guidance in catheter-lab procedures since it offers real-time volumetric imaging that enhances visualization of cardiac anatomy, compared to two-dimensional (2D) slice visualization with B-mode ultrasound, and provides exceptional soft tissue visualization, which is missing in x-ray. For many structural heart disease (SHD) interventions (e.g., mitral valve replacement), TEE is commonly used to provide visualization.

Typically, a TEE probe is inserted into the esophagus by a trained sonographer (or cardiologist) and is adjusted manually towards a number of standard viewing positions such that a particular anatomy and perspective of the heart is within the field of view of the US device. Different measurements or inspections might require different field of views/perspectives of the same anatomy, in which case the probe needs to be re-positioned. In surgery, the probe is often moved between view positions in order to accommodate X-Ray imaging.

TEE probes typically include mechanical joints that can be operated by knobs on a handle of the TEE probe. The joints, along with controlled insertion distance of the TEE probe and electronic beam steering of the ultrasound imaging plane, provides substantial flexibility in positioning the ultrasound transducer and the imaging plane so as to acquire a desired view of the heart. However, concerns include a risk of perforating the esophagus, and difficulty in manipulating the many degrees of control to achieve a desired clinical view.

In addition to TEE, other types of ultrasound imaging that employ a probe having a tube sized for insertion into a patient (i.e., a catheter) with an ultrasound transducer disposed at the distal end of the tube include: Intracardiac Echo (ICE) probes which are usually thinner than TEE probes and are inserted into blood vessels to move the ultrasound transducer array inside the heart; and Intravascular Ultrasound (IVUS) probes which are also thin and are inserted into blood vessels to image various anatomy from interior vantage points.

Many interventional procedures performed on the heart, including aortic valve repair, mitral valve repair or replacement, patent foramen ovale closure, and atrial septal defect closure, have migrated from a surgical to a transcatheter approach. In transcatheter interventions, the clinician introduces long, flexible tools into the heart through the vasculature. Transfemoral access is a common technique in which a tiny incision is made near the patient's groin to serve as an instrument portal into the femoral vein, en route to the heart.

Transcatheter approaches have risen in popularity because, compared to surgery, they impose less trauma on patients and require less postoperative recovery time. At the same time, they are technically challenging procedures to perform due to lack of dexterity, visualization, and tactile feedback. Some of these essential capabilities are restored through technologies such as TEE, which restores vision lost by minimal access approaches, and to a lesser extent replaces tactile feedback with visual feedback of the tool-to-tissue interactions.

The following discloses certain improvements to overcome these problems and others.

### SUMMARY

In one aspect, an ultrasound device comprises a probe including a tube sized for in vivo insertion into a patient and an ultrasound transducer disposed at a distal end of the tube. A camera is mounted at the distal end of the tube in a spatial relationship to the ultrasound transducer. At least one electronic processor is programmed to: control the ultrasound transducer and the camera to acquire ultrasound images and camera images respectively while the ultrasound transducer is disposed in vivo; construct keyframes during in vivo movement of the ultrasound transducer, each keyframe representing an in vivo position of the ultrasound transducer and including at least ultrasound image features extracted from at least one of the ultrasound images acquired at the in vivo position of the ultrasound transducer and camera image features extracted from at least one of the camera images acquired at the in vivo position of the ultrasound transducer; generate a navigation map of the in vivo movement of the ultrasound transducer comprising the keyframes; and output navigational guidance based on comparison of current ultrasound and camera images acquired by the ultrasound transducer and camera with the navigation map.

In another aspect, a navigation device for navigating a probe including a tube sized for in vivo insertion into a patient and an ultrasound transducer disposed at a distal end of the tube is disclosed. The navigation device includes at least one electronic processor programmed to: control the ultrasound transducer of the probe to acquire ultrasound images while the ultrasound transducer is disposed in vivo inside a patient; construct keyframes during in vivo movement of the ultrasound transducer inside the patient, each keyframe representing an in vivo position of the ultrasound transducer and including (i) at least ultrasound image features extracted from the ultrasound images acquired at the in vivo position of the ultrasound transducer, and (ii) a configuration of the probe at the in vivo position of the ultrasound transducer; generate a navigation map of the in vivo movement of the ultrasound transducer comprising the keyframes; and output navigational guidance based on comparison of a current ultrasound image acquired by the ultrasound transducer with the navigation map.

In another aspect, a method of controlling an ultrasound device comprising a probe including a tube sized for insertion into a patient and an ultrasound transducer disposed at a distal end of the tube and a camera mounted at the distal end of the tube in a fixed spatial relationship to the ultrasound transducer is disclosed. The method includes: controlling the ultrasound transducer and the camera to acquire ultrasound images and camera images respectively while the ultrasound transducer is disposed in vivo inside a patient; constructing keyframes during in vivo movement of the ultrasound transducer, each keyframe representing an in vivo position of the ultrasound transducer and including at least ultrasound image features extracted from at least one of the ultrasound images acquired at the in vivo position of the ultrasound transducer and camera image features extracted from at least one of the camera images acquired at the in vivo position of the ultrasound transducer and a configuration of the probe at the in vivo position of the ultrasound transducer, wherein the in vivo movement of the ultrasound transducer includes movement from a first view consisting of a first in vivo position of the ultrasound transducer to a second view consisting of a second in vivo position of the ultrasound transducer; generating a navigation map of the in vivo movement of the ultrasound transducer comprising the keyframes, the navigational map including a first view keyframe representative of the first view, a second view keyframe representative of the second view, and intermediate keyframes representative of intermediate positions of the ultrasound transducer during the movement from the first view to the second view; and outputting navigational guidance based on comparison of current ultrasound and camera images acquired by the ultrasound transducer and camera with the navigation map.

One advantage resides in providing proper positioning of an ultrasound probe to acquire cardiac images at specific views.

Another advantage resides in providing a catheter-based ultrasound probe with improved robotic control of the ultrasound probe, or improved navigational guidance in the case of manual operation of the ultrasound probe.

Another advantage resides in providing an ultrasound probe with spatially arranged multiple image devices (e.g., the ultrasound probe and an auxiliary camera) to provide more information for navigating the probe to different cross-sectional views.

Another advantage resides in providing an ultrasound probe with improved navigation to provide faster targeting of specific views.

Another advantage resides in providing an ultrasound probe that provides a navigational map and guidance to a user for maneuvering the ultrasound probe through a patient.

Another advantage resides in providing an ultrasound probe with less operational complexity, reducing errors and costs.

Another advantage resides in providing an ultrasound probe with servomotors and an electronic controller that automatically maneuvers the ultrasound probe through an esophagus, blood vessel, or other anatomy having a traversable lumen.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, in various steps, and arrangements of steps. The drawings are only for purposes of illustrating the preferred embodiments and are not to be construed as limiting the disclosure.

FIGS. 1 and 2 illustrate an exemplary embodiment of an ultrasound device in accordance with one aspect.

FIG. 3 shows exemplary flow chart operations of the ultrasound device of FIGS. 1 and 2.

FIG. 4 shows an example of a keyframe generated by the ultrasound device of FIGS. 1 and 2.

FIG. 5 shows potential movable axes of the ultrasound device of FIGS. 1 and 2.

FIG. 6 shows an example of keyframes and corresponding links generated by the ultrasound device of FIGS. 1 and 2.

FIG. 7 shows an example of a navigation map generated by the ultrasound device of FIGS. 1 and 2.

FIG. 8 shows an example use of the ultrasound device of FIGS. 1 and 2.

### DETAILED DESCRIPTION

The systems and methods disclosed herein utilize keyframes. As used herein, a keyframe (and variants thereof) refers to a signature of a position of the probe position. The keyframe includes at least an image signature representing a particular position of a TEE probe (or other catheter-based ultrasound probes). In some embodiments, the keyframe may be a configuration keyframe (or variant thereof) which refers to a keyframe that combines an image signature representing a particular position of the TEE probe with the corresponding TEE probe configuration (defined in terms of joint angles, tube rotation, insertion depth, image plane, and possibly other degrees-of-freedom of the TEE probe).

It is recognized herein that ultrasound images alone can be insufficient for generating reliable keyframes, because the ultrasound imaging can be intermittent and provides a relatively low-resolution image. To provide more robust keyframes, a video camera is integrated into the probe tip, attached with the ultrasound transducer or positioned closely thereto on the cable so as to move together.

In a typical workflow, the TEE probe acquires keyframes at points along the traversal of the esophagus. For example, a new keyframe may be acquired each time the image loses (due to movement and/or electronic beam steering) more than a threshold fraction of image features. Optionally, when the physician reaches a desired view a manual acquisition of a keyframe may be acquired and labeled with the view. Alternatively, the view may be recognized automatically based on image analysis automatically identifying defining image features, and the corresponding keyframe labeled with the view. In the case of a robotic TEE probe, if the physician then wants to return to a previous view, one or more servo motors are reversed to move the probe tip backwards, and the acquired images are compared with key points along the way to automatically trace and adjust (if needed) the backtracking process. In the case of a manually operated TEE probe, human perceptible guidance (e.g., text, audio) is provided to guide the operator in moving the probe tip backwards, and the acquired images are compared with key points along the way to automatically trace the backtracking process and provide updated guidance if needed based on the comparisons.

As the TEE probe traverses the esophagus and is moved into desired clinical views, in one approach configurational keyframes are acquired. From these, a navigation map is constructed, which identifies configurational keyframes and links between them. The links identify the navigational path to move from one keyframe to another. This makes it easier to return to a previous view and to verify when the previous view is reached. The navigation map may also allow for optimization of the path between two views.

In some embodiments disclosed herein, a manual mode is implemented. In this case, the TEE probe is a manually operated probe having knobs for controlling the joints of the TEE probe, and the system provides control prompts such as “advance insertion”, “retract”, “at view”, or so forth based on the route derived from the navigational map and comparison of the real-time configurational keyframes with previously-acquired configurational keyframes. In other embodiments, the TEE probe is partly or completely robotic, with servomotors replacing the knobs operating the TEE probe joints. In this case, the system can directly control the servomotors to execute the desired TEE probe manipulations.

In some embodiments disclosed herein, the ultrasound transducer is side-emitting while the video camera is forward looking, which is a convenient arrangement as a side-emitting ultrasound transducer is well-placed to image the heart, while the forward-looking video camera provides a vantage that is not provided by the side-emitting transducer. Of particular value, a forward-looking camera can detect an obstruction that would prevent further insertion of the TEE probe, and can visualize the appropriate action (e.g. turning of a probe joint) to avoid collision with the obstruction.

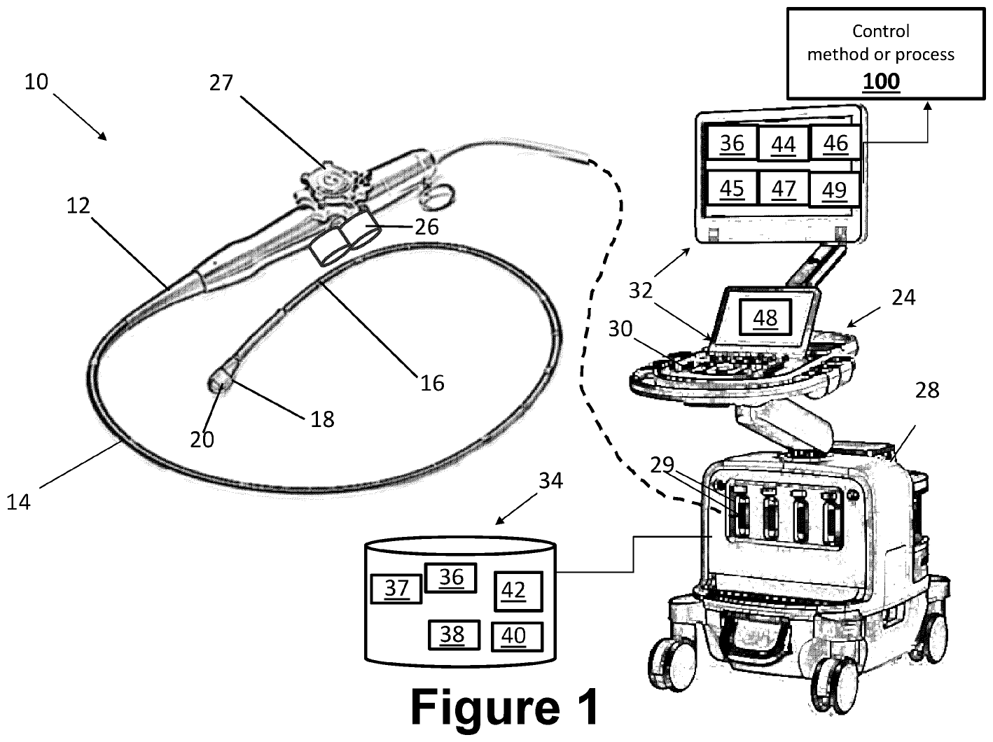

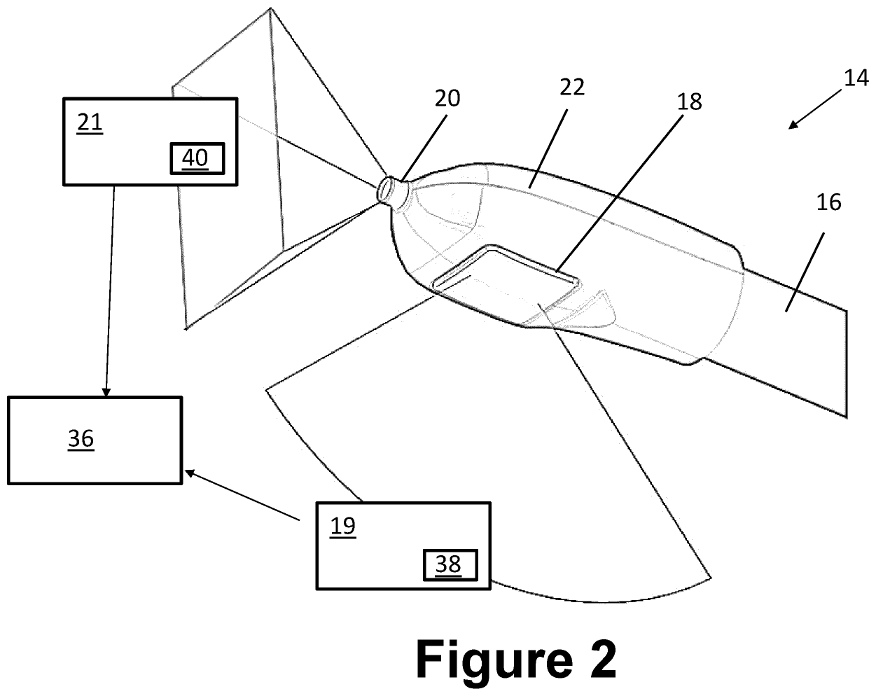

FIGS. 1 and 2 illustrate one exemplary embodiment of an ultrasound navigation device 10 for a medical procedure, in particular a cardiac imaging procedure. Although referred to herein as a TEE ultrasound device, the ultrasound device 10 be any suitable catheter-based ultrasound device (e.g., an ultrasound device for an intracardiac echo (ICE) procedure, intravascular US (IVUS) procedures, among others). As shown in FIG. 1, the ultrasound device 10 includes a probe 12 configured as, for example, a flexible cable or tube that serves as a catheter for insertion into a lumen of the patient (e.g., the lumen may be an esophageal lumen, or a blood vessel lumen, or so forth). The probe 12 can be any suitable, commercially-available probe (e.g., a Philips x7-2 TEE probe, available from Koninklijke Philips N.V., Eindhoven, the Netherlands). The illustrative probe 12 is described as being used in a TEE procedure including inserting the probe into an esophagus of a patient to acquire images of the patient's heart, but it will be appreciated that the catheter-based probe can be suitably sized to be inserted into any portion of the patient to acquire images of any target tissue. Typically, an intravascular probe for ICE or IVUS or will be of thinner diameter compared with a TEE probe, due to the narrower lumen of the narrowest blood vessels traversed during an ICE or IVUS procedure as compared with the larger lumen of the esophagus.

The probe 12 includes a tube 14 that is sized for insertion into a portion of a patient (e.g., an esophagus). The tube 14 includes a distal end 16 with an ultrasound transducer 18 disposed thereat. The ultrasound transducer 18 is configured to acquire ultrasound images 19 of a target tissue (e.g., a heart or surround vasculature). A camera 20 (e.g., a video camera such as an RGB or other color camera, a monochrome camera, an infrared (IR) camera, a stereo camera, a depth camera, a spectral camera, an optical coherence tomography (OCT) camera, and so forth) is also disposed at the distal end 16 of the tube 14. The camera 20 is configured to acquire camera (e.g., still and/or video) images 21 of the target tissue. The camera 20 can be any suitable, commercially-available camera (such as a camera described in Pattison et al., “Atrial pacing thresholds measured in anesthetized patients with the use of an esophageal stethoscope modified for pacing”, Journal of Clinical Anesthesia, Volume 9, Issue 6, 492).

The camera 20 is mounted in a spatial relationship (i.e., a fixed spatial relationship) to the ultrasound transducer 18. In one example embodiment, the ultrasound transducer 18 and the camera 20 are attached to each other, or, as shown in FIGS. 1 and 2, housed or otherwise secured to a common housing 22 located at the distal end 16 of the tube 14. In particular, as shown in FIG. 2, the ultrasound transducer 18 is arranged to be side-emitting, and the camera 20 is arranged to be forward-facing. Advantageously, this arrangement as shown in FIG. 1 provides side-emitting ultrasound transducer 18 is well-placed to image the heart, while the forward-looking video camera 20 provides a vantage (e.g., of the heart) that is not provided by the side-emitting transducer.

The ultrasound device 10 also includes an electronic controller 24, which can comprise a workstation, such as an electronic processing device, a workstation computer, a smart tablet, or more generally a computer. In the non-limiting illustrative example, the electronic controller 24 is a Philips EPIQ class ultrasound workstation. (Note that the ultrasound workstation 24 and the TEE probe 12 are shown at different scales.) The electronic controller 24 can control operation of the ultrasound device 10, including, for example, controlling the ultrasound transducer 18 and/or the camera 20 to acquire images, along with controlling movement of the probe 12 through the esophagus by controlling one or more servomotors 26 of the ultrasound device 10 which are connected to drive joints (not shown) and/or to extend and retract the tube 14. Alternatively, one or more knobs 27 may be provided by which the user manually operates the drive joints to maneuver the probe through the esophagus.

While FIG. 1 shows both knob and servomotor components 26, 27 for illustrative purposes, typically the ultrasound probe 12 will be either manual (having only knobs) or robotic (having only servomotors), although hybrid manual/robotic designs are contemplated, such as a design in which the user manually extends/retracts the tube 14 while servomotors are provided to robotically operate the probe joints.

The workstation 24 includes typical components, such as at least one electronic processor 28 (e.g., a microprocessor) including connectors 29 for plugging in ultrasound probes (a dashed cable is shown in FIG. 1 diagrammatically indicating the TEE probe 12 is connected with the ultrasound workstation 24), at least one user input device (e.g., a mouse, a keyboard, a trackball, and/or the like) 30, and at least one display device 32 (e.g. an LCD display, plasma display, cathode ray tube display, and/or so forth). The illustrative ultrasound workstation 24 includes two display devices 32: a larger upper display device on which ultrasound images are displayed, and a smaller lower display device on which a graphical user interface (GUI) 48 for controlling the workstation 24 is displayed. In some embodiments, the display device 32 can be a separate component from the workstation 24.

The electronic processor 28 is operatively connected with a one or more non-transitory storage media 34. The non-transitory storage media 34 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 24, various combinations thereof, or so forth. While shown separately from the controller 24, in some embodiments, a portion or all of the one or more non-transitory storage media 34 may be integral with the ultrasound workstation 24, for example comprising an internal hard disk drive or solid-state drive. It is to be further understood that any reference to a non-transitory medium or media 34 herein is to be broadly construed as encompassing a single medium or multiple media of the same or different types. Likewise, the electronic processor 28 may be embodied as a single electronic processor or as two or more electronic processors. The non-transitory storage media 34 stores instructions executable by the at least one electronic processor 28.

The ultrasound device 10 is configured as described above to perform a control method or process 100 for controlling movement of the probe 12. The non-transitory storage medium 32 stores instructions which are readable and executable by the at least one electronic processor 28 of the workstation 24 to perform disclosed operations including performing the control method or process 100. In some examples, the control method 100 may be performed at least in part by cloud processing.

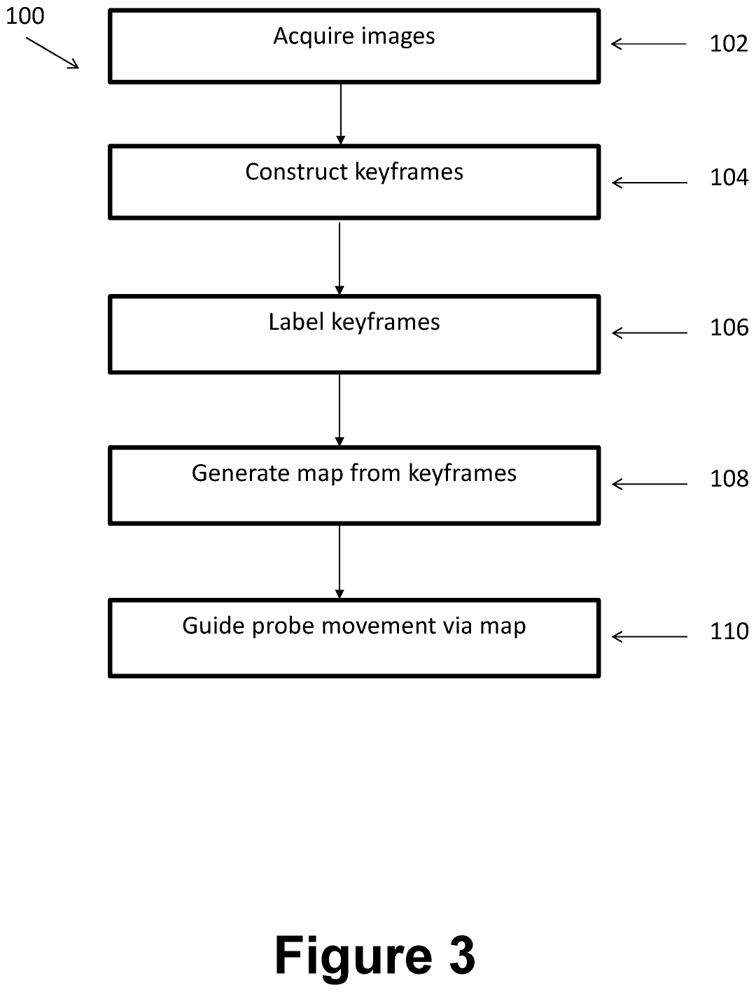

Referring now to FIG. 3, and with continuing reference to FIGS. 1 and 2, an illustrative embodiment of the control method or process 100 is diagrammatically shown as a flowchart. At an operation 102, the at least one electronic processor 28 is programmed to control the ultrasound transducer 18 and the camera 20 to acquire ultrasound images 19 and camera images 21 respectively while the ultrasound transducer (and also the camera 20 and the common rigid housing 22) is disposed in vivo inside the esophagus of the patient.

At an operation 104, the at least one electronic processor 28 is programmed to construct multiple keyframes 36 during in vivo movement of the ultrasound transducer 18. Each keyframe 36 is representative of an in vivo position of the ultrasound transducer 18 (e.g., within the esophagus). To construct the keyframes 36, the at least one electronic processor 28 is programmed to extract ultrasound image features 38 from at least one of the ultrasound images 19, and/or extract camera image features 40 from at least one of the camera images 21. The ultrasound images 19 and the camera images 21 can be stored in the one or more non-transitory computer media 34, and/or displayed on the display device 32. The extraction process can include an algorithm to extract feature sets between the at least one ultrasound image feature 38 and the at least one camera image feature 40. Such algorithms can include, for example, a scale-invariant feature transform (SIFT) algorithm, a multi-scale-oriented patches (MOPS), algorithm, a vessel tracking algorithm, or any other suitable matching algorithm known in the art. In a variant embodiment, the operation 102 acquires only ultrasound images using the ultrasound transducer 18 (in which case the camera 20 may optionally be omitted), and the operation 104 constructs the keyframe using features 38 extracted only from the ultrasound images. However, it is expected that constructing the keyframes 36 using features extracted from both the ultrasound image 19 and the camera image 21 will provide the keyframes 36 with a higher level of discriminativeness for uniquely identifying a given view, and moreover the camera image 21 can be useful in situations in which the ultrasound image has low contrast or otherwise has information-deficient features (and vice versa, if the camera image is information-deficient then this is compensated by the features extracted from the ultrasound image).

In one example, the keyframes 36 can further include features comprising a configuration 37 of the probe 12 at the in vivo position of the ultrasound transducer 18. The configuration 37 can be stored in the non-transitory computer readable medium 34, and can include one or more settings (e.g., beam steering angle, focus depth, resolution, width, and so forth) of the ultrasound transducer 18 at the acquisition time of the ultrasound image 19 from which the image feature 38 is extracted at the in vivo position of the transducer. The configuration 37 of the probe 12 can additionally or alternatively include a tube extension setting of the probe and/or joint position settings of the probe at the acquisition time of one or more of the ultrasound images 19. In a further example, the configuration 37 of the probe 12 can include an imaging plane of one of the ultrasound images 19 acquired at the in vivo position of the ultrasound transducer 18. The electronic beam steering setting of the ultrasound imaging plane provides substantial flexibility in positioning the ultrasound transducer 18 and the imaging plane so as to acquire a desired view of the heart.

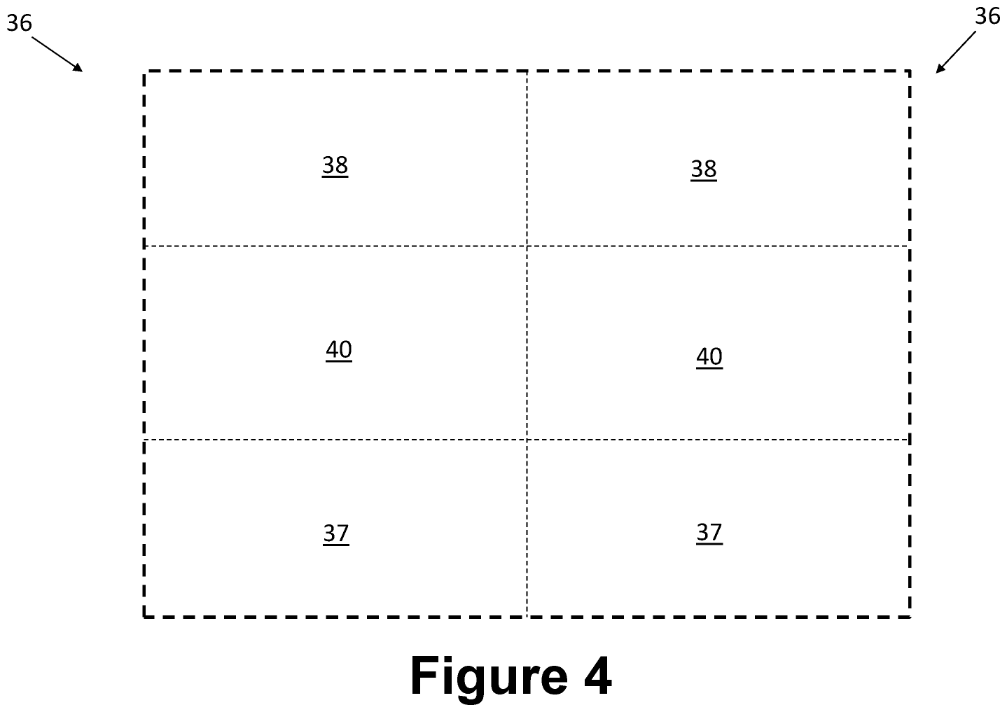

The keyframes 36 can be configured as a collection, or tuple, of information, including the ultrasound image features 38, the camera image features 40, and the settings in the configuration 37 of the probe 12. Each position of the probe 12 can be represented as a unique tuple. FIG. 4 shows an example of such a tuple of two adjacent keyframes 36. The tuple can be stored in memory (i.e., the non-transitory computer readable medium 36) as any suitable data structure, e.g. a single vector concatenating the elements of the tuple, or as a separate vector for each element of the tuple, or as a multidimensional array data structure, or so forth.

In some example embodiments, the at least one electronic processor 28 is programmed to construct a keyframe 36 that is representative of a first view consisting of a first in vivo position of the ultrasound transducer 18. During traversal of the ultrasound transducer 18 from the first view to a second view consisting of a second in vivo position of the ultrasound transducer, the at least one electronic processor 28 is programmed to construct keyframes 36 representative of “intermediate” positions of the ultrasound transducer. At the end of the traversal of the ultrasound transducer 18, the at least one electronic processor 28 is programmed to construct a keyframe 36 representative of the second view.

The at least one electronic processor 28 is programmed to detect when a new keyframe 36 representative of the “intermediate positions” should be acquired and saved (i.e., during the transition from the first view to the second view). To do so, the most recently constructed keyframes 36 is compared to the most recently-acquired ultrasound images 19 and the most recently-acquired camera images 21. In one example if the number of features (e.g., anatomical features, and so forth) in the images 19, 21 changes in a way that exceeds a predetermined comparison threshold (25% of the features) as to the number of features in the keyframes 36, a new keyframe is generated. In another example, the average pixel displacement in the acquired images 19, 21 changes by a predetermined comparison threshold (e.g., x % of the image size) relative to the pixel displacement of the keyframe 36, then a new keyframe is generated. Other examples can include deformable matching algorithms known in the art to improve the images 19, 21 to image tracking. These thresholds can be empirically tuned, e.g., for example, to ensure that a “correct” number of keyframes 36 is acquired (e.g., too many keyframes results in aliasing keyframes, while too few keyframes makes navigation difficult).

Other examples to determine when a new keyframe 36 should be acquired include: a distance from a most-previously acquired keyframe, a distance from any keyframe, a time elapsed from the most previously-acquired keyframe, a sufficient dissimilarity from last image (either ultrasound or camera), a sufficient dissimilarity from any image, a sufficient joint motion, and combinations thereof. Construction of keyframes 36 can also be triggered by a signal, such as an ECG signal, an anatomical signal (e.g. measured respiratory signal), or other synchronizing signal. The keyframes 36 may optionally further include information about any medical interventional instruments or tissue tracking information.

In other example embodiments, the operation 104 includes constructing each keyframe 36 responsive to satisfaction of one or more keyframe acquisition criteria 42 (which can be stored in the one or more non-transitory computer readable media 34). In one example, the keyframe acquisition criterion 42 can include a comparison between a “last-acquired” keyframe 36 and currently-acquired ultrasound images 19 and/or currently-acquired camera images 21. The keyframes 36 can be stored in the one or more non-transitory computer media 34, and/or displayed on the display device 32. Once stored, the keyframes 36 can be access at any time by the user via the workstation 24. The comparison can include a comparison of a change in a number of features between the last-acquired keyframe 36 and the ultrasound images 19/camera images 21, a spatial shift of one of the ultrasound images 19 or one of the camera images, with the last-acquired keyframe, and so forth. In another example, the keyframe acquisition criterion 42 can include a recognition of a defining image feature of a target tissue imaged in a current ultrasound image 19 (e.g., the left or right ventricle, the left or right aorta, a specific blood vessel of a heart of the patient, such as the aorta or vena cava, and so forth). The comparison process can include applying a matching algorithm to match the feature sets 38 and 40 of the at least one ultrasound image 19 and the at least one camera image 21, respectively. Such algorithms can include, for example, using a sum of squared differences (SSD) algorithm. In some examples, a deformable registration algorithm can be applied to the feature sets 38 and 40 to improve reliability of the matching between multiple keyframes 36. To increase the robustness of the keyframe matching, a sequence of the most recently-generated keyframes 36 are optionally used in the matching process.

In an optional operation 106, the at least one electronic processor 28 is programmed to label, with a label 44, a keyframe 36 representative of the in vivo position of the ultrasound transducer 18 upon receiving a user input from a user via the at least one user input device 30 of the workstation 24. In one approach, the GUI 48 may provide a drop-down list GUI dialog of standard anatomical views (a midesophageal (ME) four chamber view, a ME (long axis (LAX) view, a transgastric (TG) Midpapillary short axis (SAX) view, among others) and the user can select one of the listed items as the label 44. Alternatively, a free-form text entry GUI dialog may be provided via which the user types in the label 44, or further annotates a label selected from a drop-down list. In addition, keyframes 36 can also be labeled as being indicative or representative of intermediate positions of the ultrasound transducer 18 (e.g., a position of the ultrasound transducer in a position between positions shown in “adjacent” ultrasound images 19 and/or camera images 21). The labels 44 and the labeled keyframes 36 can be stored in the one or more non-transitory computer readable media 34. The labels 44 can also include, for example, corresponding events such as surgical subtasks, adverse events, and so forth.

In some examples, rather than (or in addition to) employing manual labeling, the at least one electronic processor 28 can be programmed to label or otherwise classify the ultrasound images 19 and/or the camera images 21 according to particular anatomical views shown in the images (e.g., ME four chamber view, ME LAX view, TG Midpapillary SAX view, among others). The images 19 and 21 can be manually labeled by the user via the at least one user input device 30, or automatically labeled using ultrasound image matching algorithms known in the art.

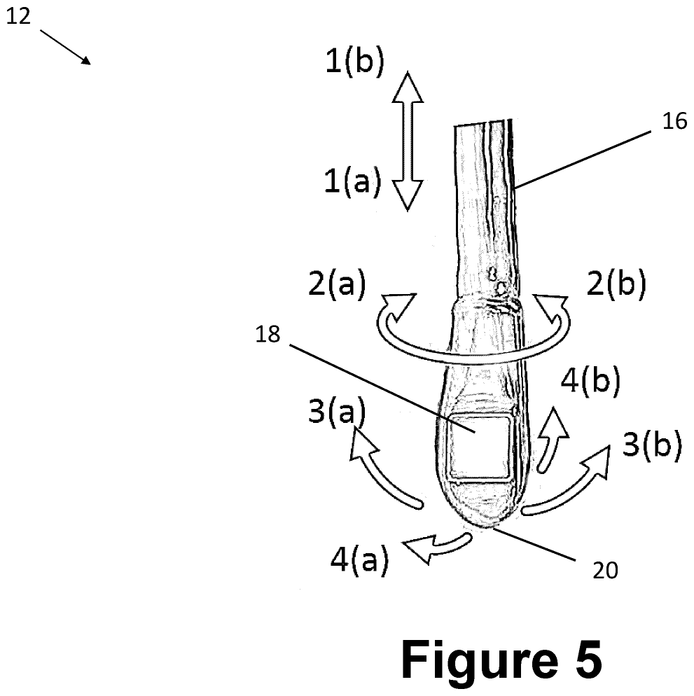

Referring briefly now to FIG. 5, and with continuing reference to FIGS. 1-3, the probe 12 is manipulatable (manually using knobs 27 or other manual manipulation, and/or robotically using servomotors 26, depending upon the embodiment) in a variety of manners. The probe 12 is able to laterally advance (labeled along a direction 1(a) in FIG. 3); laterally withdraw along a direction 1(b); rotate along a forward angle direction 2(a), and rotate along a back-angle direction 2(b). The distal end 16 of the probe 12 is configured to move (via user operation of the knobs 27) in a right direction 3(a); a left direction 3(b); an ante-flexion direction 4(a); and a retro-flexion direction 4(b). These are illustrative degrees of freedom; a specific ultrasound probe implementation may provide more, fewer, and/or different degrees of freedom for manipulating the probe position in vivo.

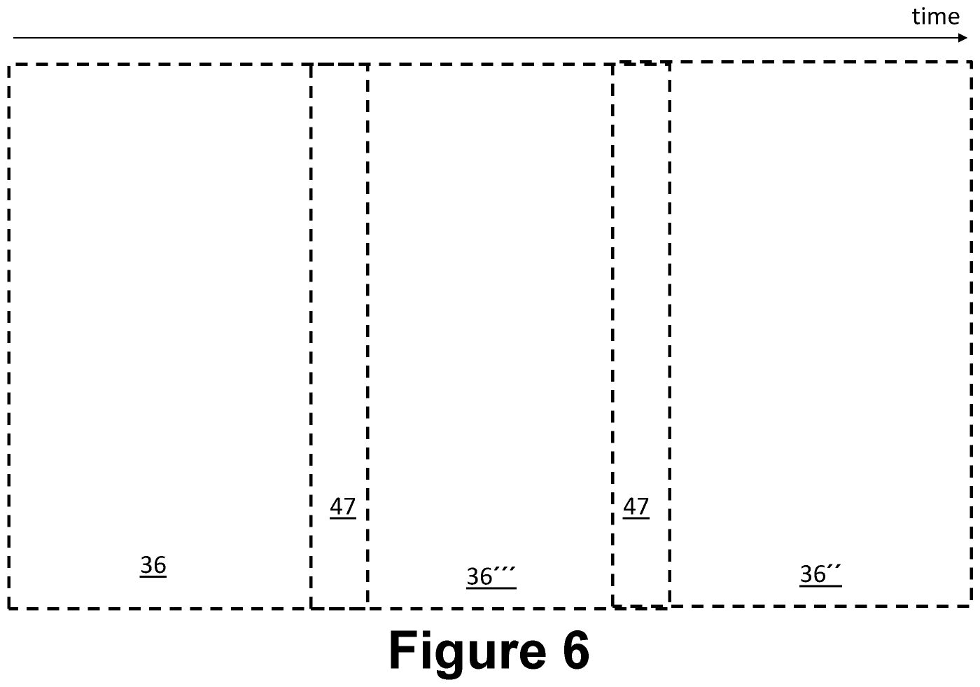

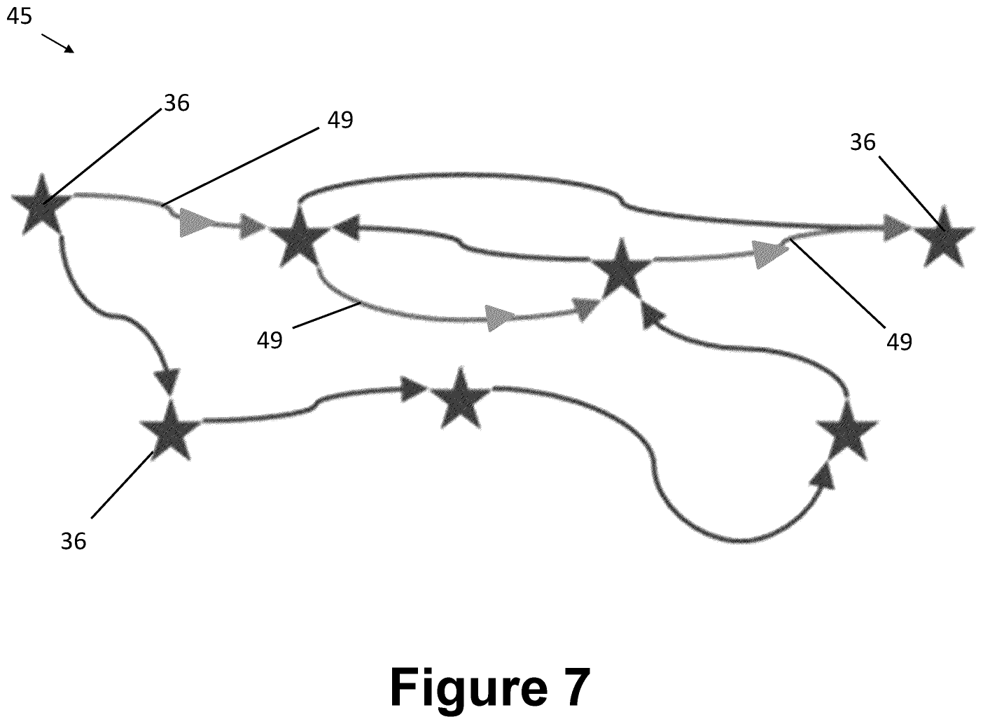

With continuing reference to FIGS. 1-3, and referring now to FIGS. 6 and 7, in an operation 108, the at least one electronic processor 28 is programmed to generate a navigation map 45 of the in vivo movement of the ultrasound transducer 18. FIG. 6 shows a portion of a time sequence of events used in constructing the navigation map 45, while FIG. 7 diagrammatically shows a navigation map 45. The navigation map 45 comprises the keyframes 36 (i.e., generated at the operation 104). To generate the navigation map 45, the at least one electronic processor 28 is programmed to identify one or more links 47 between the keyframes 36 based on a temporal sequence (FIG. 6) of the construction of the keyframes representative of the in vivo positions of the ultrasound transducer 18 during the in vivo movement of the ultrasound transducer. As shown in FIG. 6, the links 47 connect adjacent keyframes 36 (e.g., between a first view keyframe 36′ and a second key view keyframe 36″; between the first view keyframe and an intermediate keyframe 36′″; and so forth). The links 47 identify the navigational path to move from one keyframe 36 to another. For example, each link 47 may comprise a recorded time-ordered sequence of probe adjustments performed between the last keyframe and the next keyframe. This makes it easier to return to a previous view and to verify when the previous view is reached. The links 47 can be computed depending on an efficiency with which the probe 12 can be navigated towards the target tissue. The efficiency can be determined from a number of metrics, such as join displacements of the probe, a distance traveled, a force exerted by the probe, a number of intervening keyframes 36, and so forth.

When the probe 12 is in the position of keyframe 36″ shown in FIG. 6, to go from the keyframe 36″ back to the earlier (i.e., first view) keyframe 36, it would in principle be sufficient to: (i) rewind (i.e., repeat, but in reverse order) the link 47 from keyframe 36″ to the intermediate keyframe 36′; and (ii) rewind the link 47 from intermediate keyframe 36′ to the first view keyframe 36. However, in practice a simple rewind may be insufficient, for various reasons. The probe 12 may drift during movement due to forces applied on the probe by walls of the esophagus, thus altering the traversal path. The probe joints may also exhibit some hysteresis or other mechanical imperfections which can also alter the traversal path. To address, this, the electronic controller 24 suitably performs matching of the current keyframe with any available keyframes along the path (such as the illustrative intermediate keyframe 36″ shown in FIG. 6) to ensure that the rewind is progressing as intended. If deviations are identified (e.g., the current keyframe does not match the expected intermediate keyframe 36″ after performing the rewind of the first link 47), then adjustments to the probe joints or other degrees of freedom can be made to align the current keyframe with the intermediate keyframe 36″. This can be done iteratively, e.g. adjust a joint by a small amount and see if the match is improved, if not adjust the joint in the opposite direction, and iteratively repeat until a best match is obtained, then repeat this iterative optimization for another joint of the probe 12, and so forth. Alternatively, the comparison of the current keyframe with the intermediate keyframe 36″ can be used to estimate the correct direction of the adjustment, e.g. based on the shift between key features in the current keyframe compared with the expected positions of those key features in intermediate keyframe 36″. If the keyframes 36 include the configuration information, this can be used as well in making adjustments during the rewind, e.g. if the joint positions of the current frame after rewinding the first link 47 do not precisely match the configuration recorded in the intermediate keyframe 36″, then the joints can be adjusted to more closely match the keyframe configuration.

In another approach, the links 47 may not be recorded. In this case, the intermediate keyframes 36″ should be acquired at sufficiently small intervals, and preferably with the configuration information in the keyframes, so that rewind from a current keyframe to a previous keyframe can be performed by iteratively adjusting the joints or other probe degrees of freedom to step from the configuration of one intermediate keyframe to the configuration of the next intermediate keyframe, and so forth, until the configuration of the previous keyframe is reached.

The navigation map 45 may also allow for optimization of the path between two views. The navigation map 45 can be used to determine paths to previously visited locations, with the potential to reduce path redundancies and thereby increasing navigation efficiency. The navigation map 45 may also be used to extrapolate to unmapped positions based on what has been mapped. In some examples, the navigation map 45 can be updated (e.g., on the display device 30 via the GUI 48) to reflect live conditions (i.e., from inside of the esophagus).

Returning now to FIGS. 1-3, in an operation 110, the at least one electronic processor 28 is programmed to output navigational guidance 49 based on comparison of current ultrasound and camera images 19, 21 acquired by the ultrasound transducer 18 and camera 20, respectively, with the navigation map 45. The navigational guidance 49 may additionally or alternatively be based on the links 47, e.g. implementing the recorded time-ordered sequence of probe adjustments performed or a rewind of the recorded sequence. The navigational guidance 49 may additionally or alternatively be based on the stepwise changes between the configurations of successive intermediate keyframes. In the latter approaches, the navigation guidance 49 determined from the links 47 and/or stepwise changes in configurations of successive intermediate keyframes 36″ is preferably verified (and adjusted if needed) based on the comparisons of current ultrasound and camera images 19, 21 with the keyframes of the navigation map 45. For example, the at least one electronic processor 28 is programmed to guide (and, in the case of robotic embodiments, control) in vivo movement of the probe 12 through the esophagus via the construction of multiple keyframes 36 using the navigational guidance 49. The guidance 49 can be output on the display device 30 via the GUI 48.

In one example embodiment, the operation 110 is implemented in a manual mode. To do so, the at least one electronic processor 28 is programmed to provide human-perceptible guidance 46 during a manually executed (e.g. via knobs 27) backtracking traversal (i.e., “reverse” movement) of the ultrasound transducer 18 back from the second view to the first view. The guidance 46 is based on comparisons of the ultrasound images 19 and the camera images 21 (acquired during backtracking traversal) with the keyframes 36 representative of the intermediate positions and the keyframe representative of the first view. The guidance 46 can include commands including one or more of: advancement of the ultrasound device 10 through the esophagus (e.g., “go forward and variants thereof); retraction of the ultrasound device through the esophagus (e.g., “reverse” and variants thereof), “turn,” “capture a keyframe”, and so forth. The guidance 46 can be output visually on the display device 32, audibly via a loudspeaker (not shown), and so forth. In addition, the guidance 46 can be displayed as overlaying the images 19 and 21 as displayed on the display device 32.

In another example embodiment, the operation 110 is implemented in an automated mode, in which the probe 12 is automatically moved through the esophagus by action of servomotors 26. To do so, the at least one electronic processor 28 is programmed to control the one or more servomotors 26 of the probe 12 to perform the traversal of the ultrasound transducer 18 from the first view to the second view. The at least one electronic processor 28 is then programmed to control the servomotors 26 of the probe 12 to perform a backtracking traversal of the ultrasound transducer 18 back from the second view to the first view based on comparisons of the ultrasound images 19 and the camera images 21 (acquired during the backtracking traversal) with the keyframes 36 representative of the intermediate positions, and the keyframe representative of the first view.

In both the manual mode and the automated mode, the at least one electronic processor 28 is programmed to guide the user in regard to the movement of the probe 12 through the esophagus by generating the GUI 48 for display on the display device 32. The user can use the GUI 48 to select a desired view or keyframe 36 using the at least one user input device 30. The desired view of keyframe 36 can include a keyframe that was previously-acquired and stored in the non-transitory computer readable medium 34, keyframes acquired during a current procedure, or predefined keyframes stored in the non-transitory computer readable medium. The matching algorithm for the image feature sets 38, 40 can be used to find a set of keyframes 36 that is closest to a current acquired keyframe as shown on the display device 30. For example, keyframes 36 from “view A” to “view N” are created by a user at the beginning of a procedure and saved in the non-transitory computer readable media 34. The views between adjacent views (e.g., “view A” to view “B”, “view B” to “view C”, and so forth) are linked using the “intermediate” keyframes 36. To do so, incremental motion between a current keyframe (e.g., “view B”) and a next keyframe (e.g., “view C”) using, for example, a motion estimation method such as a basic optical flow of features to estimate which way the probe 12 should move. The incremental motion direction that is required to move the probe 12 to the next keyframe to a desired view is implemented on the GUI 48. The incremental motion can be presented relative to, for example, a view of the camera 20, a view of the ultrasound transducer 18, a model of the probe 12, a model of the heart, a model of the patient, and so forth. The incremental motion can be shown, for example as a three-dimensional area indicated the direction of movement.

FIG. 7 shows an example of the navigation map 45. The keyframes 36 are represented as stars, and the “single-head” arrows are representative of movement of the probe 12 through the esophagus (i.e., through each of the keyframes 36). The guidance 49 is represented as “double-head” arrows. The double-head arrows of the guidance 49 represent an optimized path for the user to guide the movement of the probe 12 through the esophagus.

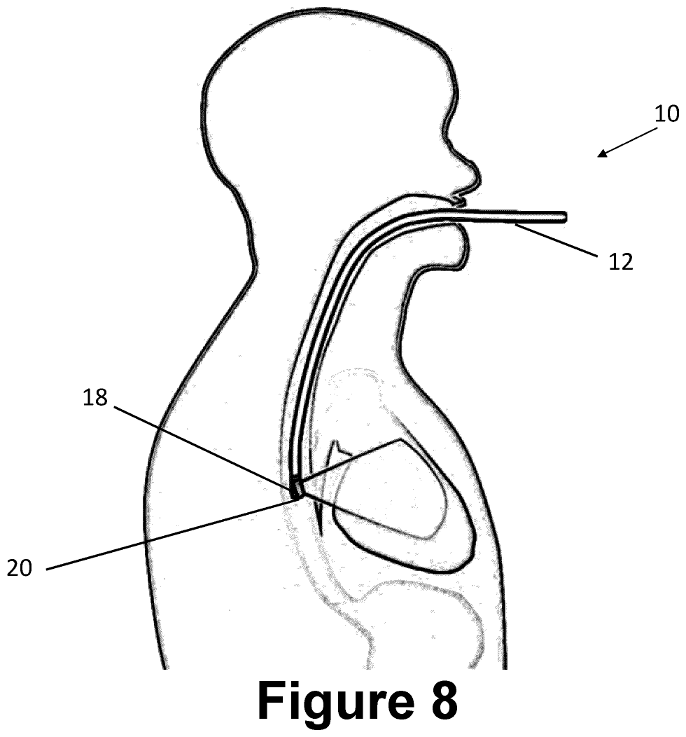

FIG. 8 shows an example use of the ultrasound device 10 inserted in vivo into a patient's esophagus. As shown in FIG. 8, the probe 12 is inserted down the esophagus of the patient so that the ultrasound transducer 18 and the camera 20 can acquire the respective ultrasound images 19 and the camera images 21 of the patient's heart. It will be appreciated that this is merely one specific application of the disclosed approaches for guiding a catheter-based ultrasound probe. For example, an Intracardiac Echo (ICE) or Intravascular Ultrasound (IVUS) probe can be analogously guided through a major blood vessel(s) of the patient to reach desired anatomical views, and to backtrack to a previous anatomical view.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An ultrasound device , comprising:
a probe including a tube sized for in vivo insertion into a patient and an ultrasound transducer disposed at a distal end of the tube;
a camera mounted at the distal end of the tube in a spatial relationship to the ultrasound transducer; and
at least one electronic processor programmed to:
control the ultrasound transducer and the camera to acquire ultrasound images and camera images respectively while the ultrasound transducer is disposed in vivo;
construct keyframes during in vivo movement of the ultrasound transducer, each keyframe representing an in vivo position of the ultrasound transducer and including at least ultrasound image features extracted from at least one of the ultrasound images acquired at the in vivo position of the ultrasound transducer and camera image features extracted from at least one of the camera images acquired at the in vivo position of the ultrasound transducer;
generate a navigation map of the in vivo movement of the ultrasound transducer comprising the keyframes; and
output navigational guidance based on comparison of current ultrasound and camera images acquired by the ultrasound transducer and camera with the navigation map.

2. The ultrasound device of claim 1, wherein the at least one electronic processor is programmed to generate the navigation map by operations including:
identifying links between the keyframes based on a temporal sequence of the construction of the keyframes representative of the in vivo positions of the ultrasound transducer during the in vivo movement of the ultrasound transducer.

3. The ultrasound device of claim 1, wherein each keyframe further includes a configuration comprising one or more settings of the probe at the acquisition time of the ultrasound image acquired at the in vivo position of the ultrasound transducer.

4. The ultrasound device of claim 3, wherein the configuration of the probe includes tube extension, tube rotation, and joint position settings of the probe at the acquisition time of the ultrasound image acquired at the in vivo position of the ultrasound transducer.

5. The ultrasound device of claim 1, wherein the ultrasound transducer and the camera are attached to each other or housed in or secured to a common rigid housing disposed at the distal end of the tube, the ultrasound transducer is arranged on the tube to be side-emitting, and the camera is arranged on the tube to be forward-facing.

6. The ultrasound device of claim 1, wherein the at least one electronic processor is programmed to construct each keyframe during the in vivo movement of the ultrasound transducer responsive to satisfaction of a keyframe acquisition criterion.

7. The ultrasound device of claim 6, wherein the keyframe acquisition criterion comprises a comparison between a last keyframe and currently acquired ultrasound and camera images.

8. The ultrasonic device of claim 6, further including at least one user input device; and wherein the at least one electronic processor is programmed to:
label the keyframe representative of the in vivo position of the ultrasound transducer upon receiving a user input via the at least one user input device.

9. The ultrasound device of claim 1, wherein the in vivo movement of the ultrasound transducer includes movement from a first view consisting of a first in vivo position of the ultrasound transducer to a second view consisting of a second in vivo position of the ultrasound transducer, and the navigation map includes:
a first view keyframe representative of the first view;
a second view keyframe representative of the second view; and
intermediate keyframes representative of intermediate positions of the ultrasound transducer during the movement from the first view to the second view.

10. The ultrasound device of claim 9, wherein the output of navigational guidance includes:
during a backtracking movement of the ultrasound transducer back from the second view to the first view, provide human-perceptible guidance for manual control of the probe based on comparisons of ultrasound images and camera images acquired during backtracking movement with the keyframes representative of the intermediate positions and the keyframe representative of the first view.

11. The ultrasound device of claim 10, wherein the human-perceptible guidance includes commands including one or more of: guidance to advance the ultrasound device, guidance to retract the ultrasound device, and guidance to adjust a joint of the probe.

12. The ultrasound device of claim 9, wherein the probe further includes servomotors, and the at least one electronic processor is further programmed to:
control the servomotors of the probe to perform the in vivo movement of the ultrasound transducer;
wherein the output of navigational guidance includes controlling the servomotors of the probe to perform a backtracking movement of the ultrasound transducer back from the second view to the first view based on comparisons of ultrasound images and camera images acquired during the backtracking traversal with the keyframes representative of the intermediate positions and the keyframe representative of the first view.

13. The ultrasound device of claim 1, wherein the probe comprises a transesophageal echocardiography (TEE) probe sized for esophageal insertion.

14. A navigation device for navigating a probe including a tube sized for in vivo insertion into a patient and an ultrasound transducer disposed at a distal end of the tube, the navigation device comprising:
at least one electronic processor programmed to:
control the ultrasound transducer of the probe to acquire ultrasound images while the ultrasound transducer is disposed in vivo inside a patient;
construct keyframes during in vivo movement of the ultrasound transducer inside the patient, each keyframe representing an in vivo position of the ultrasound transducer and including (i) at least ultrasound image features extracted from the ultrasound images acquired at the in vivo position of the ultrasound transducer, and (ii) a configuration of the probe at the in vivo position of the ultrasound transducer;
generate a navigation map of the in vivo movement of the ultrasound transducer comprising the keyframes; and
output navigational guidance based on comparison of a current ultrasound image acquired by the ultrasound transducer with the navigation map.

15. The navigation device of claim 14, wherein the at least one electronic processor is programmed to generate the navigation map by operations including:
identifying links between the keyframes based on a temporal sequence of the construction of the keyframes representative of the in vivo positions of the ultrasound transducer during the in vivo movement of the ultrasound transducer.

16. The navigation device of claim 14, wherein the in vivo movement of the ultrasound transducer includes movement from a first view consisting of a first in vivo position of the ultrasound transducer to a second view consisting of a second in vivo position of the ultrasound transducer, and the navigation map includes:
a first view keyframe representative of the first view;
a second view keyframe representative of the second view; and
intermediate keyframes representative of intermediate positions of the ultrasound transducer during the movement from the first view to the second view.

17. The navigation device of claim 16, wherein the output of navigational guidance includes:
during a backtracking movement of the ultrasound transducer back from the second view to the first view, provide human-perceptible guidance for manual control of the probe based on comparisons of ultrasound images acquired during backtracking movement with the keyframes representative of the intermediate positions and the keyframe representative of the first view.

18. The navigation device of claim 16, wherein the probe further includes servomotors, and the at least one electronic processor is further programmed to:
control the servomotors of the probe to perform the in vivo movement of the ultrasound transducer;
wherein the output of navigational guidance includes controlling the servomotors of the probe to perform a backtracking movement of the ultrasound transducer back from the second view to the first view based on comparisons of ultrasound images acquired during the backtracking traversal with the keyframes representative of the intermediate positions and the keyframe representative of the first view.

19. The navigation device of claim 14, wherein the probe further includes a camera mounted at the distal end of the tube in a fixed spatial relationship to the ultrasound transducer, and the at least one electronic processor is programmed to:
control the camera to acquire camera images while the ultrasound transducer is disposed in vivo inside a patient;
wherein each keyframe further includes camera image features extracted from at least one of the camera images acquired at the in vivo position of the ultrasound transducer; and
wherein the navigational guidance is output based on comparison of current ultrasound and camera images acquired by the ultrasound transducer and camera with the navigation map.

20. A method of controlling an ultrasound device comprising a probe including a tube sized for insertion into a patient and an ultrasound transducer disposed at a distal end of the tube and a camera mounted at the distal end of the tube in a fixed spatial relationship to the ultrasound transducer, the method comprising:
controlling the ultrasound transducer and the camera to acquire ultrasound images and camera images respectively while the ultrasound transducer is disposed in vivo inside a patient;
constructing keyframes during in vivo movement of the ultrasound transducer, each keyframe representing an in vivo position of the ultrasound transducer and including at least ultrasound image features extracted from at least one of the ultrasound images acquired at the in vivo position of the ultrasound transducer and camera image features extracted from at least one of the camera images acquired at the in vivo position of the ultrasound transducer and a configuration of the probe at the in vivo position of the ultrasound transducer, wherein the in vivo movement of the ultrasound transducer includes movement from a first view consisting of a first in vivo position of the ultrasound transducer to a second view consisting of a second in vivo position of the ultrasound transducer;
generating a navigation map of the in vivo movement of the ultrasound transducer comprising the keyframes, the navigational map including a first view keyframe representative of the first view, a second view keyframe representative of the second view, and intermediate keyframes representative of intermediate positions of the ultrasound transducer during the movement from the first view to the second view; and
outputting navigational guidance based on comparison of current ultrasound and camera images acquired by the ultrasound transducer and camera with the navigation map.
